(19) United States
(12) Patent Application Publication
(10) Pub. No.: US 2011/0009627 A1
(43) Pub. Date: Jan. 13, 2011

(54) REACTOR FOR CARRYING OUT HIGH PRESSURE REACTIONS, METHOD FOR STARTING AND METHOD FOR CARRYING OUT A REACTION

(75) Inventors: Helmut Schmidtke, Bensheim (DE); Wilhelm Ruppel, Mannheim (DE); Helmut Berrsche, Hassloch (DE); Johann-Peter Melder, Bohl-Iggelheim (DE); Udo Rheude, Otterstadt (DE); Petr Kubanek, Mannheim (DE); Gerhard Ruf, Frankenthal (DE); Wolfgang Gunkel, Mannheim (DE); Wolfgang Mägerlein, Mannheim (DE); Karl-Heinz Ross, Grunstadt (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/864,540

(22) PCT Filed: Jan. 21, 2009
(86) PCT No.: PCT/EP2009/050642

§ 371 Date: Jul. 26, 2010

(30) Foreign Application Priority Data
Jan. 25, 2008 (EP) 08150677.6
Jul. 16, 2008 (EP) 08160471.2

(51) Int. Cl. B01J 19/00; C07D 265/30; C07C 209/04

(52) U.S. Cl. 544/106; 422/240; 422/119; 422/198; 564/474; 564/478

(57) ABSTRACT

The invention relates to a reactor for performing high-pressure reactions, comprising at least one tube (31) whose ends are each conducted through a tube plate (33) and which is bonded to the tube plate (33). The tube plates (33) and the at least one tube (31) are surrounded by an outer jacket, such that an outer space (39) is formed between the tube (31) and the outer jacket. The tube plates (33) each have at least one surface composed of a nickel-base alloy and the at least one tube (31) is in each case welded on to the surface composed of the nickel-base alloy. The surface composed of the nickel-base alloy points in each case in the direction of the particular reactor end. The outer jacket has a thickness which is sufficient to absorb tensile forces which occur between tube (31) and outer jacket owing to a temperature difference in the event of different expansion. The invention further relates to a process for starting up the reactor and for performing an exothermic reaction in the reactor.

The invention relates to a reactor for performing high-pressure reactions, comprising at least one tube whose ends are each conducted through a tube plate and which is bonded to the tube plate, the tube plates and the at least one tube being surrounded by an outer jacket, such that an outer space is formed between the tube and the outer jacket. The invention further relates to a process for starting up the reactor, the at least one tube being filled with a catalyst which is activated by a reduction with hydrogen. Finally, the invention also relates to a process for performing an exothermic reaction in the reactor.

Currently, high-pressure reactions are generally performed in adiabatic high-pressure furnaces. These are insulated from the environment, and the temperature profile which is established arises from the conversion of reactants. If, however, for example as a result of outage in the power supply, the supply of the reactants can no longer be ensured, there can be uncontrolled runaway in some reactions. This can lead especially to a high temperature increase. This may be accompanied by a high evolution of pressure, which can lead to bursting of the reactor.

A further disadvantage of the high-pressure reactors known from the prior art is that, owing to variations caused by regulation, a varying temperature distribution occurs in the inlet to the high-pressure reactor and hence the composition of the output likewise has variations.

Thick-walled vessels for adiabatic reactions at pressures up to 1000 bar are described, for example, in S. Maier, F. J. Müller, “Reaktionstechnik bei industriellen Hochdruckverfahren”, CIT 58, 1986, p. 287 to 296. Typical applications are hydrogenations and aminations at 200 bar and coal hydrogenation at 700 bar. For even higher pressures, a long thin high-pressure tube with wall cooling is used, which, however, is unsuitable for heterogeneously catalyzed syntheses.

Compared to adiabatic high-pressure furnaces, tube bundle reactors have the advantage that a virtually isothermal temperature profile can be established along the tube axis, and hence better exploitation of the catalyst bed and very low-variation synthesis conditions are possible. A high pressure on the inside of the tube is, however, unfavorable, since the tubes are stressed on a bend. Very massive hoods and tube plates are required, which leads to the result that the reactor is very expensive or no longer implementable in the manner of construction customary to date. In practice, the use of tube bundle reactors is therefore currently restricted to the medium-pressure range up to 100 bar.

In order also to utilize the advantages of a tube bundle reactor at high pressure, U.S. Pat. No. 4,221,763, for example, describes a reactor with a high-pressure vessel, into which a tube bundle is installed. In this case, the coolant should in each case have the same pressure as the pressure in the tube interior, such that the internals may also be of filigree structure. How the pressure is equalized between tube interior and coolant side is, however, not stated.

This principle is also known from ammonia synthesis. In the so-called “Leuna furnace”, as described, for example, in H. Buchter, “Apparate and Armaturen der Chemischen Hochdrucktechnik” [Apparatus and Fittings from Chemical High-Pressure Technology], Springer-Verlag 1967, Ch. VI, section III, pages 240 to 254, a tube bundle is disposed in a high-pressure vessel. The catalyst is accommodated in the tubes. Preheated synthesis gas flows around the tubes as a coolant. There is thus only a small pressure difference between tube interior and tube exterior, which is determined by the pressure drop at the catalyst bed. The function is thus also ensured in the case of variations in the operating pressure, which is typically at 221 bar.

It is an object of the present invention to provide a reactor for performing high-pressure reactions, which has a homogeneous temperature profile and hence does not have the disadvantages of the high-pressure reactors known from the prior art. It is a further object to provide a reactor for performing high-pressure reactions which can be operated safely even in the event of outage in the power supply.

The object is achieved by a reactor for performing high-pressure reactions, comprising at least one tube whose ends are each conducted through a tube plate and which is bonded to the tube plate, the tube plates and the at least one tube being surrounded by an outer jacket, such that an outer space is formed between the tube and the outer jacket. The tube plates each have at least one surface composed of a nickel-base alloy. The at least one tube is in each case welded on to the surface composed of the nickel-base alloy, the surface composed of the nickel-base alloy pointing in each case in the direction of the particular reactor end. The outer jacket has a thickness which is sufficient to absorb tensile forces which occur between tube and outer jacket owing to a temperature difference in the event of different expansion.

In the context of the present invention, a high-pressure reaction means that apparatus and machines are designed for a pressure range from 100 to 325 bar. The reaction can be effected within this pressure range or else at a pressure of less than 100 bar.

The advantage of the outer jacket which has a thickness which is sufficient to absorb tensile forces which occur between tube and outer jacket owing to a temperature difference in the event of different expansion is that no compensator has to be provided in the outer jacket. Such a compensator typically has the form of bellows which can be compressed and extended in axial direction and which are accommodated in the outer jacket. However, such a compensator has the consequence that a compressive force which acts on the outer jacket cannot be absorbed by it, since the compensator deforms. By virtue of dispensing with the compensator, it is possible to absorb compressive forces acting on the outer jacket. These arise, for example, through the mass of the tube plates and the covers of the reactor.

The thickness of the outer jacket depends on how great is the maximum temperature difference which occurs between the tube interior and the outer jacket. The greater the temperature difference, the more different is the longitudinal expansion owing to the temperature. In the case of an exothermic reaction, the tubes are generally hotter than the outer jacket. This means that the tubes expand to a higher degree than the outer jacket. As a result, bending occurs through the tube plate. This leads to a tensile force which acts on the outer jacket. This tensile force has to be absorbable by the outer jacket.

The necessary thickness of the outer jacket is also dependent on the diameter of the reactor and the length of the tubes. The greater the diameter of the reactor and the longer the tubes are, the thicker the outer jacket has to be.

For example, in the case of a diameter of the tube plate of 2.35 m and a tube length of 12 m, a minimum wall thickness of the outer jacket of 25 mm is determined for a temperature difference of up to 30 K between the tube interior and the temperature on the outside of the outer jacket. In the case of a temperature difference between the tube interior and outside of the outer jacket of up to 45 K, for example, a wall thickness of at least 35 mm is determined, and a temperature difference of up to 60 K requires, for example, a wall thickness of the outer jacket of at least 70 mm. In the case of a correspondingly smaller reactor diameter or shorter tubes with the same temperature difference, the thickness of the outer wall can be selected in each case at a somewhat lower level. Correspondingly, the wall thickness of the outer jacket would have to be selected at an even higher level in the case of a greater reactor diameter or longer tubes.

In a preferred embodiment, the nickel-base alloy is applied to the tube plates as a plating. The advantage of applying the nickel-base alloy as a plating to the tube plates is that this allows the tube plates to be manufactured from any desired material with cost advantages. The tube plates are preferably manufactured from low-alloy, thermally stable steels. The advantage of the use of low-alloy, thermally stable steels for the tube plates is that they can be forged more easily than a nickel-base alloy, and the manufacture of the tube plate is thus simplified. In contrast to the low-alloy, thermally stable steels, the nickel-base alloy is easier to weld, such that the tubes can be welded in more easily as a result of the nickel-base alloy. The plating of the nickel-base alloy is applied typically by welding, rolling or explosive plating. For this purpose, the nickel-base alloy is applied to the tube plate, for example, as a weld additive in the form of weld beads. In order to achieve the desired thickness of the plating, welding application can also be effected in several layers. After the application of the nickel-base alloy, the surface is preferably smoothed, for example by suitable grinding processes.

Suitable carbon steels from which the tube plate can be manufactured are, for example, 12 CrMo 9-10 and 24 CrMo 10 (material designation according to DIN). In addition to carbon steels, also suitable for producing the tube plate are in principle, however, stainless steels, for example X6 CrMoNiTi 17-12-2 and X3 CrNiMoN 17-13-5, in the case of which plating with nickel-base materials is dispensed with and the tubes are welded directly on the plate.

Suitable nickel-base alloys are, for example, NiCr21 Mo and NiCr15Fe.

The plating preferably has a thickness of up to 30 mm. The thickness of the plating depends on the thickness of the tube walls and the depth of the weld seam which arises therefrom. The thickness of the plating is selected such that the height of the weld roots is less than the thickness of the plating. This ensures that the weld seam does not reach into the material of the tube plate. A further advantage of this plating thickness is that an exchange of the tubes is enabled without the plating having to be renewed or without heat treatment of the apparatus to dissipate the stresses occurring through the welding-in having to be carried out.

The at least one tube used in the reactor preferably has a length in the range from 3000 to 18 000 mm. The tube length selected depends on the velocity of the reaction medium and the desired residence time. The greater the residence time should be and the higher the velocity is, the longer the tubes have to be. The reactor designed in accordance with the invention also allows a construction of the outer jacket without compensator in the case of a tube length of up to 18 000 mm.

Depending on the desired throughput and the number of individual tubes in the reactor needed for this purpose, the tube plate preferably has a diameter of up to 2400 mm and a thickness of up to 600 mm. The diameter of the tube plate arises from the number of tubes to be used in order to achieve the desired throughput. The advantage of a plurality of tubes with a comparatively small diameter in contrast to one tube with a high diameter is that, in a tube with a relatively small diameter, better temperature control by a temperature control medium which flows on the tube exterior is possible. In the case of a relatively high tube diameter, it would be necessary, if appropriate, to provide a heat exchanger within the tube. However, this is complicated in construction terms.

The thickness of the tube plate depends on the diameter of the tube plate and the length of tubes used. Since different thermal expansions of outer jacket and tubes at different temperatures cause a force to be exerted on the tube plate, it has to be sufficiently strong to be able to absorb the forces acting on it. Thus, the thickness of the tube plate must especially be selected such that it does not deform owing to its own weight alone.

The material used for the at least one tube is preferably an austenitic material or a ferritic-austenitic material. Suitable austenitic materials are, for example, X6 CrNiMoTi 17-12-2, X3 CrNiMoN 17-13-5 and X2 CrNiMoN 25-22. A suitable ferritic-austenitic material is, for example, X 2CrNiMoN 22-5-3. The advantage of austenitic materials is that they are generally very ductile and corrosion-resistant. For this reason, austenitic materials can be deformed very readily. This leads to no damage to the tubes occurring even in the case of tensile or compressive stress on the tubes, for example owing to different thermal expansion owing to temperature differences between tube interior and outer jacket. The corrosion resistance prevents the tubes from becoming weakened owing to corrosion. The advantage of the ferritic-austenitic material is that it additionally possesses an increased strength. In addition to austenitic materials, however, nickel-base materials such as NiCr21 Mo and NiCr15 Fe are, for example, also suitable as a material for the tubes.

In the case of reactions which are carried out in the presence of a heterogeneous catalyst, the tubes are filled with the catalyst which is generally present in socover form. The catalyst may, for example, be present in the form of a bed, as a random packing or as structured packing. When the catalyst is present in the form of a granule or random packing bed, at least one tray is preferably provided in the tube, on which the catalyst rests. The tray is designed, for example, as a perforated tray or as a sieve tray.

In order to enable simplified removal of the catalyst from the at least one tube, it is preferred to support the catalyst filling by means of spring elements. The spring elements used are preferably pressure springs designed as spiral springs, which are preferably designed in conical form. The spring elements also enable, for example, the catalyst to be removed at the bottom in the case of a vertical reactor, by removing the base of the reactor. This allows simpler removal of the catalyst than removal by suction from the top of the reactor.

If the reaction which is carried out in the reactor is not carried out in the presence of a heterogeneous catalyst, or the catalyst is entrained with the reaction mixture, it is alternatively also possible to provide internals for flow homogenization in the individual tubes. Such internals may, for example, also be a random packing bed or a structured packing, but it is alternatively also possible that the internals provided in the tube are, for example, trays. Such trays are, for example, perforated trays or sieve trays. In the case of a polyphasic reaction mixture, especially a gas-liquid mixture, it is necessary that the internals or the catalyst are configured such that the gas bubbles present are not accumulated as a result of the catalyst or the internals. Division of the bubbles and hence a homogeneous gas distribution in the liquid is, however, desirable.

In a particularly preferred embodiment, the outer space of the reactor is connected to a temperature control medium circuit. The temperature control medium circuit preferably comprises a reservoir vessel for the temperature control medium. In this case, the reservoir vessel is arranged at least at such a height that the temperature control medium can flow through the outer space of the reactor owing to the hydraulic pressure of the liquid. The advantage of arranging the reservoir vessel in such a way that the temperature control medium can flow through the outer space owing to the hydraulic pressure is that temperature control of the reactor is also possible in the event of outage in the power supply, for example when a pump can no longer be operated. For example, the temperature control medium can cool the reactor in order to prevent uncontrolled runaway of the reaction. The arrangement of the reservoir vessel is preferably such that the liquid level in the reservoir vessel is at least at the same height as the liquid level in the outer space of the reactor.

The temperature control medium circuit can be configured as a closed circuit or as an open circuit. Especially when the temperature control media used are, for example, coolants or thermal oils, preference is given to using a closed circuit. When water is used as the temperature control medium, it is also possible to use an open circuit. In the case of an open circuit, the temperature control medium from the reservoir vessel is conducted through the outer space of the reactor and released from the circuit from there. The temperature control medium can then, for example, be collected in a collecting vessel or, for example, be used as heating medium in a heat exchanger. When the temperature control medium used is, for example, water and it evaporates in the outer space of the reactor owing to the heat absorbed from the reaction, the water vapor formed can be used, for example, as steam for further processes. When the steam is not required, it is also possible to release it, for example, to the environment. In the case of a closed temperature control medium circuit, it is preferred when a heat exchanger is attached to the reactor, in which the temperature control medium is cooled again before it is collected again in the reservoir vessel.

The reservoir vessel is preferably of such a size that sufficient temperature control medium is present in the event of outage in the power supply to enable cooling of the reactor to an uncritical temperature.

In order to be able to adjust the flow profile in the outer space of the reactor, it is preferred when internals are arranged in the outer space. Suitable internals are, for example, perforated plates and/or deflection plates. However, also suitable as internals are, for example, random packings or structured packings or any other trays. Also suitable and preferred is the use of support grids. These have the advantage that they have a very low pressure drop in flow direction. When the temperature control medium evaporates in the outer space of the reactor owing to the heat absorbed from the reaction, it is preferred when the internals are configured such that vapor bubbles which form are not accumulated as a result of the internals.

When the flow of the temperature control medium in the outer space of the reactor is homogenized by using perforated plates, the distance between the individual perforated plates is preferably from 400 to 700 mm. In particular, the distance between the perforated plates is 500 mm.

When a pump is used in the temperature control medium circuit in order to achieve forced circulation of the temperature control medium, the pump is preferably a free-running pump. The advantage of using a free-running pump is that, in the case of a power outage, the temperature control medium from the reservoir vessel can flow through the pump without it offering any great resistance to the temperature control medium which leads to a high pressure drop. As a result, the flow of the temperature control medium through the reactor is also ensured in the case of a power outage. Suitable free-running pumps are, for example, pumps with a withdrawn impeller. Also suitable is any pump known to those skilled in the art which has a free cross section such that the pump can be flowed through where it is not actuated.

To monitor the temperature, it is preferred when thermocouples are arranged inside the at least one tube and on the outer jacket. The thermocouples can be used to detect the temperature in the interior of the tube and the temperature at the outer jacket. From this, the temperature difference can be formed. Since the reactor, especially the thickness of the outer jacket of the reactor, is preferably designed for a maximum temperature difference between the tube interior and the outer wall of the outer jacket, it is possible by means of the thermocouples to monitor this temperature difference. In the case that the maximum permissible temperature difference is approached or the maximum permissible temperature difference is exceeded, it is possible, for example, to conduct more temperature control medium through the outer space of the reactor in order to achieve greater cooling of the tubes and hence cooling in the tube interior. Alternatively, it would also be possible to reduce the temperature difference between the interior of the at least one tube and the outer jacket by means of controlled heating of the outer jacket, in order to arrive again within the range of the permissible temperature difference. This is necessary, since the reactor can be damaged in the case of expedience of the maximum permissible temperature difference.

In order to enable a sufficiently high throughput, the reactor is preferably a tube bundle reactor. In the context of the present invention, a tube bundle reactor is understood to mean a reactor having at least two tubes. Typically, a tube bundle reactor, however, has at least five tubes. The maximum number of tubes depends on the outer diameter of the tubes and the diameter of the tube plates and hence the diameter of the reactor. The greater the diameter of the reactor and the smaller the diameter of the tubes, the more tubes may be present.

In order to prevent some tubes from being flowed through by the reaction medium to a greater extent and some to a lesser extent, it is preferred to provide internals in the intake region of the tubes of the tube bundle reactor, by means of which the reactants supplied are distributed uniformly between the tubes. Known internals for homogenizing the distribution of the reactants between the individual tubes are, for example, porous sinter plates, perforated plates and sieve trays. It is also possible to use guide plates by which an incident gas jet is divided into individual bubbles or jets. It is also possible, for example, to use ring distributors. However, particular preference is given to homogenizing the distribution of the reactants by using a distributor apparatus in which a distributor plate arranged horizontally in the apparatus comprises an active surface with passage orifices and an edge which extends downward, and the distributor plate does not extend over the entire cross section of the reactor. The distributor apparatus can be supplemented by a second distributor plate which is arranged between the feed orifice for the reactants and the first distributor plate. The second distributor plate also comprises an active surface with a multitude of passage orifices and an edge which extends downward. The second distributor plate functions essentially as a preliminary distributor. Such a distributor apparatus is known, for example, from WO 2007/045574.

In general, the reactor is used in such a way that the at least one tube proceeds in vertical direction. For this purpose, it is customary to use the reactor in an apparatus framework. The reactor is typically accommodated hanging freely in the apparatus framework, such that an upper cover and a lower cover with which the reactor is sealed are freely accessible. By removing the upper cover or the lower cover, it is, for example, possible to exchange the catalyst present in the at least one tube.

In order to obtain a homogeneous temperature in the tube interior and to prevent the reaction medium which flows through the tubes from heating very greatly toward the middle of the tube compared to the tube wall, preference is given to using tubes with an internal diameter in the range from 30 to 150 mm. Particular preference is given to using tubes with an internal diameter in the range from 35 to 50 mm, for example of 42.7 mm. In order to obtain a sufficiently good heat transfer between the tube interior and the temperature control medium in the outer space with simultaneously sufficient strength of the tubes, preference is given to using tubes with a wall thickness of from 5 to 15 mm, especially with a wall thickness of from 7 to 11 mm, for example with a wall thickness of 8.8 mm. A large number of tubes can be accommodated in the reactor when they are accommodated in triangular pitch. In the case of use of tubes with an external diameter of 60.3 mm, the axis separation is, for example, 75.4 mm.

The inventive reactor is suitable especially for performing reactions at temperatures in the range from 130 to 300° C., especially in the range from 150 to 270° C. Reactions which can be performed in the inventive reactor are, for example, the preparation of aminodiglycol and morpholine, the synthesis of polyetheramines, of C₁-C₄-alkylamines, and the synthesis of cyclododecanone. It is advantageous that these reactions can be performed by using the inventive reactor at relatively high temperatures, and it is thus possible, for example, also to use less active and generally less expensive catalysts.

The invention further relates to a process for starting up the reactor. In this case, the at least one tube of the reactor is filled with a catalyst which is activated by a hydrogenation with hydrogen. The temperature control medium used is water and the heat is supplied with the aid of water vapor. The process comprises the following steps:
(a) heating the catalyst to a temperature in the range from 120 to 170° C. at a pressure in the range from 120 to 170 bar in the presence of a nitrogen atmosphere at a rate of from 5 to 15 K/h and simultaneously increasing the temperature of the in the outer space by supplying steam and increasing the pressure, such that the boiling point of the water in the outer space corresponds to the temperature inside the tube,
(b) supplying hydrogen until a concentration of hydrogen of from 1 to 3% by volume has been attained and holding the atmosphere for a period of from 5 to 8 h, then increasing the hydrogen concentration to from 4 to 6% by volume and holding the atmosphere for a period of from 5 to 8 h,
(c) increasing the hydrogen concentration to from 8 to 12% by volume and holding the concentration until the temperature in the reactor bed remains essentially constant, then increasing the hydrogen concentration to from 45 to 55% by volume,
(d) increasing the pressure inside the at least one tube to from 150 to 250 bar and increasing the temperature of the hydrogen-comprising gas passed through the tubes to from 200 to 230° C. at a rate of from 5 to 15 K/h and increasing the temperature in the outer space by supplying steam and increasing the pressure, such that the boiling point of the water in the outer space corresponds to the temperature in the tube,
(e) replacing the water-steam mixture in the outer space with dry saturated water vapor,
(f) increasing the temperature in the tube interior to from 250 to 300° C. at a rate of from 2 to 8 K/h and holding the temperature for a period of from 20 to 30 h,
(g) lowering the temperature in the tube interior to from 80 to 120° C. at a rate of from 5 to 15 K/h and simultaneously lowering the temperature in the outer space by lowering the pressure.

The activation of the catalyst is divided into a primary activation comprising steps (a) to (c), and a secondary activation comprising steps (d) to (g).

For the primary activation, the catalyst is first heated in the presence of a nitrogen atmosphere at a rate of from 5 to 15 K/h, for example at a rate of 10 K/h, to a temperature in the range from 120 to 170° C., for example to a temperature of 150° C., at a pressure in the range from 120 to 170 bar, for example at a pressure of 150 bar. At the same time, the temperature of the water in the outer space is increased. The temperature of the water in the outer space is increased by supplying steam and increasing the pressure, such that the boiling point of the water in the outer space corresponds to the temperature in the interior of the tube. At a temperature of 150° C., the pressure in the outer space is accordingly 4.76 bar. In a next step, the nitrogen atmosphere is enriched with hydrogen by supplying hydrogen until a concentration of hydrogen of from 1 to 3% by volume, for example of 2% by volume, has been attained. This atmosphere is maintained for a period of from 5 to 8 h, for example for a period of 6 h. Thereafter, the hydrogen concentration is increased to from 4 to 6% by volume, for example to 5% by volume, by supplying further hydrogen. This atmosphere is likewise maintained for a period of from 5 to 8 h, for example over a period of 6 h.

In a further step, the hydrogen concentration is increased further to from 8 to 12% by volume, for example to 10% by volume. This concentration is maintained until the temperature in the reactor bed remains essentially constant. This means that no significant temperature peaks in the reactor bed occur. A significant temperature peak is understood to mean a local higher temperature of at least 20 K compared to the mean temperature in the reactor bed. As soon as the temperature in the reactor bed remains essentially constant, the hydrogen concentration is increased further to from 45 to 55% by volume, for example to 50% by volume.

In the context of the present invention, essentially constant temperature is understood to mean that the temperature deviates from a mean temperature by not more than 5 K.

The primary activation is then followed by the secondary activation. For this purpose, the pressure in the interior of the at least one tube is first increased to from 150 to 250 bar, for example to 200 bar. In addition, the temperature of the hydrogen-comprising gas passed through the tubes is increased to from 200 to 230° C., for example to 220° C., at a rate of from 5 to 15 K/h, for example at a rate of 10 K/h. At the same time, the temperature in the outer space is raised further by supplying steam and increasing the pressure, such that the boiling point of the water in the outer space corresponds to the temperature in the tube. At a temperature in the tube of 220° C., the pressure in the outer space is thus 23.2 bar. As soon as this state has been attained, the water/steam mixture present in the outer space is replaced by dry saturated water vapor. The use of the dry saturated water vapor allows the temperature in the outer space to be increased further by superheating of the water vapor without the pressure rising further significantly. This has the advantage that the outer jacket need not be designed for greater pressures. The wall thickness of the outer jacket can be kept at a relatively low thickness. The higher the pressure would rise in the outer space, the thicker the outer jacket would otherwise have to be. A relatively thin outer jacket leads, however, to a high material saving and hence also to a weight and cost saving.

After the replacement of the water/steam mixture by dry saturated water vapor, the temperature in the tube interior is increased at a rate of from 2 to 8 K/h, for example at a rate of 5 K/h, to from 250 to 300° C., for example to 280° C. This temperature is maintained for a period of from 20 to 30 h, for example for 24 h. By virtue of the relatively slow increase in the temperature in the tube interior, the dry saturated water vapor in the outer space is also heated. By virtue of convective heat transfer and by virtue of thermal radiation, the outer jacket is also heated. This ensures that the temperature does not exceed the desired target value between interior of the tube and outer jacket. This is necessary, in order that the outer reactor jacket is not damaged owing to different thermal expansions of the tubes and of the outer jacket owing to different temperature.

Finally, the temperature in the tube interior is lowered at a rate of from 5 to 15 K/h, for example at a rate of 10 K/h. Simultaneously, the temperature in the outer space is also lowered by lowering the pressure. As a result of lowering of the pressure, the boiling point of the water vapor present in the outer space falls. It condenses out. The boiling point of the water is established in each case. As a result of lowering of the pressure, the boiling point falls, such that a controlled temperature regime is possible in the outer space of the reactor. Depending on the reaction to be carried out in the reactor, the temperature in the tube interior is lowered preferably to from 80 to 120° C., for example to 100° C. At a temperature of 100° C., water boils at ambient pressure, such that the pressure in the outer space of the reactor has likewise been lowered to ambient pressure. When a lowering to a lower temperature is desired, it would be necessary to correspondingly evacuate the outer space of the reactor in order to achieve homogeneous cooling.

When a hydrogenation with hydrogen is carried out as the reaction in the reactor, it is preferred, on completion of the activation of the catalyst, to replace the nitrogen still present in the gas circulation system with hydrogen.

In a preferred embodiment, the activation of the catalyst is preceded by performance of cleaning of the outer space. The cleaning preferably serves simultaneously to phosphatize the metallic surfaces of the outer space. In this way, the surfaces are passivated to increase the corrosion resistance.

To clean the outer space, it is first filled with deionized water. The water preferably has a temperature in the range from 20 to 50° C. The water is subsequently seeded with from 0.001 to 0.004 kg, for example 0.002 kg, of a passivating agent per kilogram of water. This is followed by heating to from 110 to 150° C., for example to a temperature of 130° C., at a rate of from 5 to 15 K/h, for example at a rate of 10 K/h. To this end, the pressure in the outer space is also increased to the boiling pressure of the water at the corresponding temperature to which the water is heated. At a boiling point of 130° C., the pressure is thus increased to 2.7 bar. The temperature is increased by feeding in steam.

The heated aqueous solution comprising passivating agent is circulated over a period of from 20 to 30 h, for example over a period of 24 h. Subsequently, the solution is cooled at a rate of from 5 to 15 K/h, for example at a rate of 10 K/h, to a temperature in the range from 90 to 100° C., for example to a temperature of 100° C. Finally, the solution is discharged by supplying an inert gas.

In a next step, the outer space is filled with deionized water having a temperature in the range from 80 to 100° C. This is seeded with from 0.0005 to 0.004 kg, for example with 0.001 kg, of a passivating agent per kilogram of water. Subsequently, heating is again effected to a temperature in the range from 110 to 150° C., for example to 130° C., at a rate of from 5 to 15 K/h, for example at a rate of 10 K/h. This solution is circulated over a period of from 20 to 30 h, for example over a period of 24 h. Subsequently, cooling is again effected at a rate of from 5 to 15 K/h, for example at a rate of 10 K/h, to a temperature in the range from 90 to 110° C., for example to a temperature of 100° C. Finally, this solution is also discharged by supplying an inert gas.

Suitable passivating agents are, for example, an alkali metal or alkaline earth metal phosphate, such as trisodium phosphate Na₃PO₄ or triammonium citrate (NH₄)₃C₆H_{S}O₇. Particular preference is given to using Na₃PO₄.

The above step is, if appropriate, repeated until the concentration of iron ions in the solution at the end of the circulation exhibits an asymptotic profile. This means that, at first, more iron ions go into the solution and the proportion of iron ions which go into solution becomes ever smaller. The cleaning passivates the surface of the tubes, tube plates and of the outer jacket in the outer space, in order that they do not corrode.

As soon as the concentration of iron ions in the solution exhibits an asymptotic profile, the outer space is flushed with deionized water having a temperature in the range from 70 to 100° C. by circulating it for a period of from 0.5 to 2 h, especially over a period of 1 h. This operation is, if appropriate, repeated by exchanging the deionized water until an electrical conductivity of the water at the end of the flushing operation of not more than 20 μS/cm is measured. The small conductivity indicates that no extraneous ions are present any longer in the water.

In addition, the invention also relates to a process for performing an exothermic reaction in the inventive reactor. In this case, at least one reactant as the reaction medium is added to the at least one tube and reacts in the tube at least partly to give a product. A temperature control medium is added to the outer space and the temperature control medium is evaporated by absorbing heat at essentially constant temperature, such that the reaction is performed under essentially isothermal conditions.

As a result of the evaporation, a liquid/vapor mixture forms in the outer space of the reactor. At a constant pressure, evaporation proceeds at a constant temperature, provided that liquid is still present. In this way, a constant temperature can be established in the outer space of the reactor. Since heat is released from the tubes to the temperature control medium in the outer space, the temperature control medium evaporates in the outer space. The tubes can be adjusted to an essentially equal temperature. Within the tubes, a temperature profile occurs from the tube axis to the tube wall. Owing to the release of heat at the tube wall, the temperature decreases from the middle of the tube toward the tube wall. The heat which is evolved in the exothermic reaction is absorbed by the temperature control medium.

In the context of the present invention, essentially isothermal conditions mean that the temperature in the interior of the tube is increased by not more than 6 K, preferably by not more than 3 K.

The advantage of the essentially isothermal reaction conditions is that the catalyst present in the at least one tube is used over the entire length flowed through under virtually identical reaction conditions. At the same predetermined reaction exit temperature, this leads to a higher conversion of the reactant to the product than, for example, in the case of adiabatic shaft reactors of conventional design. By virtue of the homogeneous exhaustion of the catalyst activity, a higher use time up to a necessary change of the catalyst is additionally associated.

A further advantage of the essentially uniform, virtually isothermal temperature level lies in the uniform composition of the discharge from the reactor.

Owing to the essentially uniform composition of the discharge from the reactor, very fine control adjustments to downstream workup apparatus, for example workup columns, can be made. For this purpose, it is possible, for example, to minimize the energy use in distillation systems used for workup and the product loss in the case of discharge of undesired secondary components from individual process stages.

The reactants are preferably supplied to the reactor from the bottom. The internals for homogenizing the distribution of the reactants between the tubes ensure a homogeneous supply into the individual tubes of the reactor. The reactants are supplied from below especially when at least one of the reactants is present in gaseous form, since the gaseous reactants generally ascend in the liquid. Even if the resulting product is gaseous, it is preferred to supply the reactants to the reactor from below.

The temperature control medium for adjusting the temperature in the tubes is likewise preferably supplied to the outer space of the reactor from below. In this way, the temperature control medium and the reaction medium are conducted through the reactor in cocurrent. The advantage of supplying the temperature control medium into the outer space of the reactor from below is that the vapor which is evolved as heat is absorbed and, associated with this, the temperature control medium evaporates ascends in the reactor. In this way, it is possible that the water vapor flows through the outer space of the reactor more rapidly than the liquid water. The water vapor can be discharged at the upper end of the outer space of the reactor. In the case of flow of the temperature control medium from the top downward, it would be necessary that the water flow entrains the vapor bubbles which evolve. This would necessitate rapid flow through the reactor. Especially in the event of outage in the power supply, this could lead to the falling velocity of the water not being sufficient to entrain the vapor bubbles. This would lead to vapor bubbles possibly evolving inside the outer space of the reactor, in which no heat can be removed from the tubes. In the case of a strongly exothermic reaction, this could lead to strong heating in the tube and thus possibly even to burnthrough of the reactor.

According to the invention, in the event of outage in the power supply, the temperature control medium from the reservoir vessel is passed through the outer space of the reactor owing to the hydraulic pressure. Since the reservoir vessel is arranged in accordance with the invention such that the liquid level of the temperature control medium in the reservoir vessel is at least essentially at the same height as the liquid level in the outer space of the reactor, temperature control medium flows from below into the reactor and evaporates along the tubes owing to the heat absorbed, and the water vapor ascends in the reactor. The water vapor which evolves is withdrawn from the reactor in order to keep the pressure essentially constant, or, in order to lower the temperature further, also to lower the pressure further. The evaporating water lowers the liquid level in the reactor, such that new temperature control medium is replenished from the reservoir vessel. In order to ensure that sufficient temperature control medium flows in even in the case of a completely flooded outer space of the reactor, it is also possible to arrange the reservoir vessel such that the liquid level in the reservoir vessel is always above the liquid level in the reactor.

In the event of outage in the power supply, the pressure in the outer space is preferably lowered. As a result of the lowered pressure in the outer space, the boiling point of the temperature control medium also falls. In this way, the temperature in the tubes can be lowered. In the ideal case, lowering to a temperature at which the reaction is ended may even be possible.

The reactor designed in accordance with the invention and the process according to the invention are suitable especially for preparing aminodiglycol and morpholine. In this case, diethylene glycol and ammonia are added as reactants to the reactor. They are converted to aminodiglycol and morpholine.

The preparation of aminodiglycol and morpholine by reaction of diethylene glycol and ammonia in the presence of hydrogen is described, for example, in WO-A 2007/036496.

The reaction is generally carried out in the presence of a hydrogen-activated heterogeneous catalyst. A suitable catalytically active material comprises, for example, before the treatment with hydrogen, oxygen compounds of aluminum and/or of zirconium, of copper, of nickel and of cobalt.

Over the catalysts known for the aminodiglycol and morpholine synthesis, as well as the formation of aminodiglycol and morpholine, it is also possible for exothermic fragmentation reactions to occur. These are generally the decomposition of diethylene glycol. The decomposition of diethylene glycol forms carbon monoxide with subsequent formation of methane and water in the presence of hydrogen, and with subsequent formation of carbon dioxide and carbon in the absence of hydrogen. The reactions are both exothermic. In addition, the decomposition of diethylene glycol in the absence of hydrogen, unlike the decomposition reaction of diethylene glycol in the presence of hydrogen, builds up pressure. In general, the two reactions can proceed simultaneously. The presence of hydrogen alone is still not sufficient to rule out the evolution of the pressure-increasing reaction, in which the diethylene glycol is decomposed and the carbon monoxide which forms reacts further to give carbon dioxide and carbon.

In the steady-state operation of the aminodiglycol and morpholine synthesis, the two decomposition reactions of diethylene glycol have no disadvantages relevant to plant safety. However, these reactions can become dominant in the event of stoppage of the ammonia used for the amination. The ammonia can be stopped, for example, in the event of outage in the power supply. In conventional, essentially adiabatic shaft reactors, as currently used in the synthesis of diethylene glycol and ammonia to give aminodiglycol and morpholine, in the event of stoppage of the ammonia used for the amination, self-accelerated, exothermic and pressure-increasing reactions can arise. These lead to dangerous and uncontrollable states. Compliance with operating parameters with which the decomposition reactions of the diethylene glycol are converted to an uncritical order of magnitude leads generally to a reduced throughput of diethylene glycol, or to a lower production rate of morpholine and aminodiglycol. In addition, compliance with the appropriate parameters in production plants cannot reliably be implemented, since the properties of the heterogeneous catalysts can change with regard to the decomposition of diethylene glycol within the use period. The advantage of use of the inventive reactor in the synthesis of aminodiglycol and morpholine is that it is intrinsically safe. Nor in the operating range relevant in production terms are any restrictions or any aids to ensure the ammonia supply required. In addition, owing to the homogeneous utilization of the catalyst activity owing to the essentially isothermal operation of the reactor, the operating costs can be lowered.

To synthesize aminodiglycol and morpholine by reaction of diethylene glycol with ammonia in the presence of hydrogen, the temperature in the interior of the at least one tube is preferably from 150 to 250° C., more particularly from 160 to 220° C. The temperature control medium used is preferably water. The pressure in the outer space of the reactor is selected such that the boiling state of water is attained in the outer space of the reactor. This means that the pressure in the outer space of the reactor is selected such that the water boils at the reaction temperature. In the reservoir vessel, in which the water which serves as the temperature control medium is stored, the boiling pressure, just like in the outer space of the reactor, is preferably in the range from 4.76 to 86 bar (abs), more particularly in the range from 6.2 to 23.2 bar (abs).

In the case of any power outage which may occur, in which case there is the threat of uncontrolled runaway of the reaction, the temperature control medium present in the reservoir vessel, generally the water, flows into the reactor. The water evaporates in the reactor and thus absorbs heat from the individual tubes in which the reaction is carried out. In the case of an open circuit, the water vapor which evolves is released to the environment. For this purpose, a valve or any other device for adjusting the pressure is opened such that the pressure in the outer region of the reactor is lowered. As a result of the lowered pressure, the boiling point of the temperature control medium also falls, as a result of which the tubes are more highly cooled from the outside. This likewise leads to cooling in the interior of the tubes. The apparatus used to adjust the pressure is preferably one which opens automatically in the event of power outage, in order that the pressure is lowered. The apparatus is preferably designed such that, in the event of outage in the electrical power supply, the reactor is decompressed to ambient pressure within a period of from 40 to 75 min with a gradient of from 80 to 120 K/h, for example with a gradient of 100 K/h. Within the same period, the reaction medium present in the tubes is also cooled to less than 150° C. This allows the morpholine and aminodiglycol synthesis to be converted to a state which is safe for a long period.

The flow through the reactor in the event of outage in the power supply, i.e. especially when the pump of the temperature control circuit also fails, is enabled by virtue of the pump used being a free-running pump. The free-running pump enables through-flow even when it is not operated. The flow then proceeds owing to the pressure of the liquid.

In the case of use of the inventive reactor to prepare aminodiglycol and morpholine from diethylene glycol and ammonia, the diethylene glycol and the ammonia are introduced as reactants, preferably from below, into the individual tubes comprising the catalyst. The amount of reactants is preferably selected such that the reactor can be operated with a catalyst hourly space velocity of from 0.5 to 4 kg of reactant per liter of reaction volume per hour, preferably with a catalyst hourly space velocity of from 1.2 to 3 kg of reactant per liter of reaction volume per hour.

In addition to the preparation of aminodiglycol and morpholine by reaction of diethylene glycol with ammonia in the presence of hydrogen, the reactor designed in accordance with the invention is also suitable for the synthesis of any other chemical compounds in the case of which self-accelerating, exothermic and pressure-increasing reactions can occur in the event of failure of equipment. In addition, the reactor designed in accordance with the invention is also suitable for use in the case of reactions in which a homogeneous composition of the reactor effluent leads to advantages in the workup, for example the distillative workup, of the products of value.

Further chemical reactions for which the reactor designed in accordance with the invention is used advantageously are, for example, the synthesis of polyetheramines, ethylamines, propylamines and butylamines by reaction of the respective alcohol with ammonia in the presence of hydrogen, and the synthesis of cyclododecanone (oxidation of cyclododecatriene with N₂O).

When the reactor configured in accordance with the invention is used for the synthesis of propylamines or butylamines, the temperature at which the reaction is performed is preferably in the range between 200 and 270° C., especially in the range between 220 and 250° C. The pressure at which the reaction is performed is preferably in the range between 10 and 250 bar, especially in the range between 50 and 150 bar.

Polyetheramines are synthesized typically by aminating polyether alcohols. The amination of polyether alcohols is performed preferably at a temperature in the range from 170 to 240° C., especially at a temperature in the range from 180 to 230° C., and a reaction pressure of preferably from 80 to 220 bar, especially from 120 to 200 bar.

Monoalcohols which can be converted to polyetheramines by amination are preferably those of the general structure (I):

R¹—X—OH (I)

where X represents

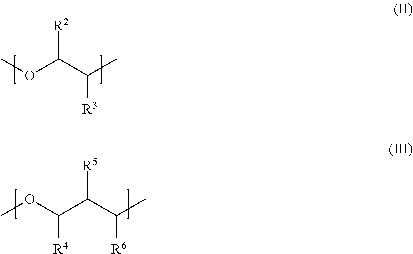

and/or
units. The two units II and III are each present in a number of from 0 to 50 in the polyether monoalcohol and are arranged in any sequence.

R¹ is C₁-C₃₀-alkyl which may be linear or branched. The R², R³, R⁴, R⁵ and R⁶ radicals are the same or different and are each independently H or linear C₁-C₁₀-alkyl.

The units (II) and/or (III) present in the polyether monoalcohol may each have identical or different substitutions.

Preference is given to using polyether monoalcohols in which only units (II) occur, where R² is preferably hydrogen and R³ is hydrogen or linear C₁-C₁₀-alkyl.

When the polyetheramines are prepared by using polyetherdiols, preference is given to using those which are propylene oxide- and/or ethylene oxide- and/or butylene oxide- and/or pentylene oxide-based. However, it is also possible that, in the polyetherdiols used to prepare the polyetheramines, the ether oxygens are bridged by an alkylene group composed of three or more carbon atoms. Suitable diols which can be used to synthesize polyetheramines are, for example, those of the general formulae IV, V and VI.

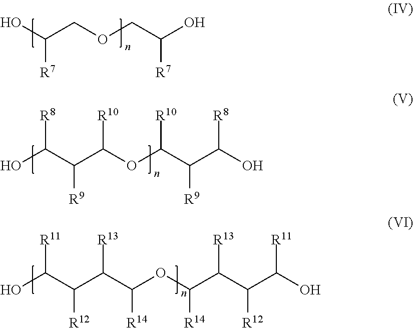

In these structures, n is in each case an integer from 1 to 50, R⁷ is hydrogen or linear C₁-C₁₀-alkyl and R⁸ to R¹⁴ are the same or different and are each independently hydrogen or methyl. It should be noted that, for example, in the general formula IV,

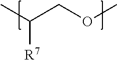

units with identical or different R⁷ radicals occur, in which case units with different substitution are present in any sequence and repetition in the particular polyetherdiol. The same applies analogously to the polyetherdiols with the

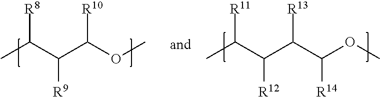

units for the R⁸ to R¹⁴ radicals.

In addition, polyetheramines can also be synthesized by using polyethertriols. The polyethertriols are preferably those of the general formula VII.

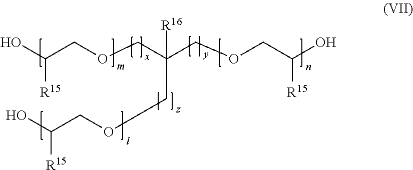

In this structure, m, n and I are the same or different and are each independently an integer from 1 to 50, x, y and z are the same or different and are each independently 0 or 1, where generally at most one of the three coefficients x, y and z is 0. R¹⁵ is hydrogen or linear C₁-C₁₀-alkyl, and R¹⁶ is hydrogen or linear or branched C₁-C₁₀-alkyl. When repeat units with different R¹⁵ radicals occur within the formula VII, the sequence and repetition of the repeat units is as desired.

The temperature control medium used is preferably water. The advantage of water is that it is not harmful to the environment when it is released to the environment as water vapor, for example in the event of outage in the power supply. A further advantage of the use of water is, for example, that the water vapor formed in the temperature control of the reactor can be utilized as steam for other processes.

Alternatively, it is, however, also possible to use, as the temperature control medium, any other temperature control medium known to those skilled in the art. For example, it is possible when the reaction medium is to be heated, for example in the course of startup of the reactor, to use a thermal oil. Alternatively, it is also possible, for example, to use a coolant, for example a mixture of water and ethylene glycol, when the reactor is to be cooled to low temperatures. In the case of use of temperature control media other than water, it is preferred when the temperature control medium circuit is operated closed, in order that no temperature control medium can escape to the environment. In the case of outage in the power supply, it is preferred in this case when an apparatus for lowering the pressure is provided, which is accommodated in a line which opens into a collection vessel. The temperature control medium is then collected in the collection vessel and thus does not get into the environment.

One embodiment of the invention is shown in the figures and is described in detail in the description below.

The figures show:

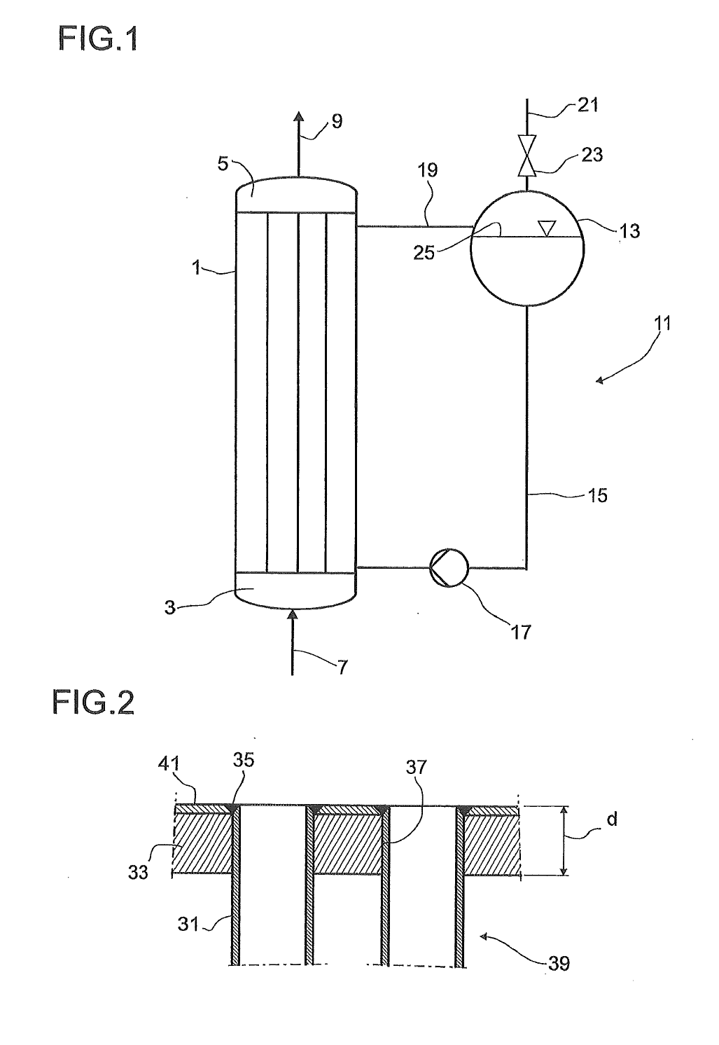

FIG. 1 a flow diagram of a reactor designed in accordance with the invention,

FIG. 2 a section of a tube plate with tubes secured therein.

FIG. 1 shows a flow diagram of a reactor designed in accordance with the invention with a temperature control circuit.

A reactor 1 is preferably designed as a tube bundle reactor. Alternatively, it is, however, also possible that, for example, a single tube with a jacket is used. The tubes of the reactor 1 are secured by each end in a tube plate. This enables, firstly, flow through the tubes, and, on the other hand, it is also possible for a second medium to flow around the tubes. In order for flow around the tubes to be possible, the reactor 1 is surrounded by an outer jacket which surrounds the tubes.

The tubes open firstly into a distributor space 3 and secondly into a collection space 5. In the distributor space 3, medium which is to be conducted through the tubes is distributed between the individual tubes. In order to achieve a homogeneous distribution, it is preferred when internals which ensure homogeneous distribution between the tubes are provided in the distributor space 3. Suitable distributors are, for example, distributor plates which have, on an active surface, passage orifices and an edge which extends downward. The distributor plate does not extend over the entire cross section of the distributor space 3. In addition to the distributor plate, a preliminary distributor may be provided. This is of the same design as the distributor plate and has, on an active surface, passage orifices and an edge which extends downward. Typically, the preliminary distributor has a smaller diameter than the distributor plate. In addition to the distributor plates, it is also possible to use any other distribution apparatus known to those skilled in the art.

The securing of the tubes in the tube plate prevents medium from the distributor space from flowing past the tubes. To this end, the tubes are mounted in the tube plate in a gas- and liquid-tight manner. This is preferably done by welding the tubes into the tube plate.

The medium added via the distributor space 3 then flows through the tubes and leaves them in the collection space 5. The collection space 5 is likewise delimited by a tube plate on the side on which the tubes open. On this side too, the tubes are secured in the tube plate in a gas- and liquid-tight manner. This is also done on the side of the collection space 5 preferably by welding the tubes into the tube plate.

In order to supply the medium which flows through the tubes, at least one feed 7 opens into the distributor space 3. When a synthesis is carried out in the reactor 1, the reactants needed for the synthesis are supplied via the feed 7. Alternatively, it is also possible to provide a dedicated feed 7 for each reactant which is supplied to the reactor 1. In this case, the mixing is effected in the distributor space 3. However, it is preferred first to mix the reactants and to feed them to the distributor space 3 together via a feed 7. Typically, the reactants fed to the reactor 1 are liquid or gaseous. Mixtures of liquids and gases are also possible. In addition, it is also possible that, for example, socover finely distributed in a liquid is fed to the reactor 1 via the feed 7. The socover dispersed in the liquid may, for example, be a reactant or else a heterogeneous catalyst. When a heterogeneous catalyst is used, it is, however, preferably accommodated in a fixed manner in the tubes. The catalyst may, for example, be present in pulverulent form, as a granule, as a random packing bed or as a structured packing. Depending on the reaction to be carried out, a suitable catalyst is used. In this case, the catalyst may consist either only of the catalytically active material, or a supported catalyst is used. In this case, the catalytically active material is bonded to a carrier substance.

In particular, the reactor 1 designed in accordance with the invention is suitable for performing reactions which are carried out in the presence of a catalyst which has to be activated before the start of the reaction. The activation can, for example, be effected by reaction with hydrogen. To this end, the catalyst disposed in the tubes is first heated, and a hydrogen atmosphere is passed through the catalyst. Typically, hydrogen-enriched nitrogen is used for this purpose. This is supplied via the feed 7, flows through the catalyst-filled tubes, collects in the collection space 5 and is discharged from the reactor 1 via an outlet 9 which opens into the collection space 5. A temperature equalization between the tubes and the outer jacket is effected via a temperature control circuit 11. Through the temperature control circuit 11, a temperature control medium which flows around the tubes is fed to the reactor. The temperature control medium, for example, absorbs heat from the tubes and releases some of it to the outer jacket. Typically, the heat required for the activation is supplied by heating the hydrogen-comprising gas stream which is supplied via feed 7 before entry into the reactor 1. The temperature in the reactor 1 is kept at hydrogenation temperature by means of the temperature control circuit 11. When heat is released in the course of activation of the catalyst, it can, for example, be absorbed by the temperature control medium present in the temperature control circuit 11. A further advantage of the temperature control circuit 11 is that the temperature control medium releases heat from the tubes of the tube bundle reactor 1 to the outer jacket which is heated as a result. As a result of this, significantly different expansions of the tubes and of the outer jacket owing to different temperatures are prevented. This enables the use of an outer jacket without an additional compensator.

After the activation operation has ended, the reactants needed for the reaction to be carried out in the reactor 1 are supplied via the feed 7. In the reactor, the conversion to the desired product is effected. The product, unconverted reactant and any by-products formed are collected in the collection space 5 and discharged from the reactor via the outlet 9. The outlet 9 is, for example, connected to a workup device. Suitable workup devices are, for example, a distillation plant in which the substances present in the output are separated from one another in order to obtain a pure product. In addition to a distillation system, however, it is also possible to use any other workup device. When the reactants are converted completely in the reactor, it is also possible to directly collect the product present in the outlet 9 or, if appropriate, to use it as a starting substance for a further reaction.

In order to be able to perform the reaction in the reactor 1 in a controlled manner, especially in the case of an exothermic reaction, it is necessary to remove the heat which evolves. This is done by means of the temperature control circuit 11. The temperature control circuit 11 comprises a reservoir vessel 13 in which the temperature control medium is initially charged. The temperature control medium is generally liquid. A feed line 15 is used to feed the temperature control medium to the reactor 1. The feed for the temperature control medium is configured such that the temperature control medium flows around the tubes present in the reactor from the outside. For the transport of the temperature control medium, a pump 17 is incorporated in the feed line 15. With the aid of the pump 17, the temperature control medium is transported into the reactor. The reaction in the reactor 1 is preferably carried out under isothermal conditions. In order to be able to realize this, the temperature control medium evaporates at least partly during the flow through the outer space of the reactor 1. The vapor-liquid mixture which arises is fed back to the reservoir vessel 13 via an outlet 19. Alternatively, it is also possible, especially when the temperature control medium used is water, to release the heated temperature control medium directly from the temperature control circuit. In this case, the circuit is an open circuit. When the temperature control medium is not fed to the reservoir vessel 13 via the outlet 19 but rather discharged from the temperature control circuit, it is necessary to supply a corresponding amount of new temperature control medium to the reservoir vessel 13. Alternatively, however, it is also possible to utilize the reservoir vessel 13 to separate vapor and liquid and to remove the vapor from the reservoir vessel 13 via a steam line 21. The vapor can, for example, be used as steam for further processes. In this case too, it is necessary to supply water to the reservoir vessel 13 in the amount in which vapor is removed. When the temperature control medium is not water but rather any other temperature control medium, for example a thermal oil or a coolant, it is preferred when a heat exchanger in which the temperature control medium is cooled before being fed into the reservoir vessel 13 is present in the outlet 19. Alternatively, it is also possible, for example, to provide a cooling coil in the reservoir vessel 13, such that the reservoir vessel 13 simultaneously functions as a heat exchanger. By means of the heat exchanger, the heat which is absorbed during passage through the reactor 1 is released again.

In order to ensure that the reaction carried out in the reactor 1 does not run away in the event of outage in the power supply, it is preferred to use a free-running pump as the pump 17. This enables flowthrough in the case of stoppage of the pump. This is required especially in the event of a power outage, since the pump 17 cannot be actuated in such an event. Owing to the hydraulic pressure, the liquid temperature control medium from the reservoir vessel 13 flows through the feed line 15 into the outer space of the reactor 1. In the reactor 1, the temperature control medium evaporates and thus cools the tubes. In order to achieve the desired temperature at which the temperature control medium boils, it is possible to adjust the pressure in the temperature control circuit 11, for example by means of a valve 23 which is present in the vapor line 21. When, for example, the temperature control medium used is water and the vapor line 21 opens into the environment, it is possible to establish ambient pressure in the outer space of the reactor 1 through the valve 23. In this case, the water used as the temperature control medium boils at a temperature of 100°. This allows the reaction in the tubes to cool down to this temperature. If cooling to a temperature below 100° C. is desired, it is necessary to use, as the temperature control medium, a temperature control medium which has a lower boiling point at ambient pressure.

In addition to the valve 23 in the vapor line 21, for the establishment of the pressure, it is also possible to provide, for example, a valve in the region of the outlet 19, by means of which the vapor which evolves can be discharged from the temperature control circuit 11. In this case, the pressure is adjusted by means of the additional valve which is not shown here. Preferably, the cooling causes cooling of the reaction medium in the tubes to a temperature at which the reaction is stopped, or it is necessary to cool until all reactants still present in the tubes have reacted. This is sufficient especially in the case when the reactant supply via the feed 7 can be stopped even in the case of power outage. For this reason, the amount of temperature control medium present in the reservoir vessel 13 is selected such that cooling-down of the reactor 1 can be ensured in any case. In order to enable flow through the reactor 1 even in the case of outage of the power supply, i.e. in the case of a stationary pump 17, the reservoir vessel 13 is preferably mounted at a height such that the liquid level 25 of the temperature control medium present in the reservoir vessel 13 is at least at the same height as the liquid level of the temperature control medium in the outer space of the reactor 1. Alternatively, it is also possible, especially when the vapor which evolves is not recycled into the reservoir vessel but rather released to the environment, to position the reservoir vessel 13 such that the liquid level 25 in the reservoir vessel 13 is higher than the liquid level in the outer space of the reactor 1.

FIG. 2 shows a section of a tube plate with tubes secured therein.

The tubes 31 of the reactor 1 are secured by each end in a tube plate 33. The tubes 31 are secured in the tube plate 33 generally with the aid of a weld seam 35. To this end, passage orifices 37 are formed in the tube plate 33, through which the tubes 31 are conducted. With the aid of the weld seam 35, each tube 31 is secured by positive locking in the tube plate 33, in which case the weld seam 35 is simultaneously a gas- and liquid-tight bond, in order that no reaction medium from the distributor space 3 or collection space 5 can penetrate through the passage orifice 37 between tube 31 and tube plate 33 into the outer space 39 of the reactor 1. At the same time, this also prevents temperature control medium from the outer space 39 from passing through the passage orifice 37 between tube 31 and tube plate 33 into the distributor space 3 or collection space 5.

The tube plate 33 is preferably manufactured from a low-alloy, thermally stable steel. The thickness d of the tube plate 33 depends on the diameter of the reactor 1 and the length of the tubes 31. Typically, the thickness d of the tube plate 33 is up to 600 mm.

According to the invention, the tube plate 33 is provided with a plating 41. The plating 41 is preferably manufactured from a nickel-base alloy. To apply the plating 41 to the tube plate 33, the nickel-base alloy is preferably applied by an application welding process, rolling or explosive plating. To this end, the nickel-base alloy is welded on to the tube plate 33 as a weld additive. Depending on the thickness of the plating, which depends on the thickness of the walls of the tubes 31 and hence the depth of the weld seam 35, the nickel-base alloy is applied in several layers. The thickness of the plating 41 is typically up to 20 mm. A flat surface is achieved by initially applying the plating in a greater thickness and then bringing it to the final thickness by suitable methods. For ablation and homogenization of the surface 41, milling and grinding processes, for example, are suitable.

The material used for the tubes 31 is preferably an austenitic material, a high-alloy steel or a nickel-base material. The latter has a sufficiently high ductility to avoid damage to the tubes 31 through thermal expansion and stresses occurring owing to different thermal expansions of tubes 31 and outer jacket. The tubes 31 are welded into the plating 41.

The plating 41 composed of the nickel-base alloy serves, especially in the case of use of aggressive media as the reaction medium, as corrosion protection. A further advantage of the plating composed of the nickel-base alloy is that, in the case of exchange of reactor tubes 31, they can be welded in again in a simple manner, without the reactor having to be annealed to balance out stresses, since the base material, especially the low-alloy, thermally stable steel of the tube plate 33 is not melted by the welding-in of the tube 31.

The outer jacket of the reactor 1 is preferably welded to the tube plate 33. Alternatively, a flange ring can be mounted on the outer jacket of the reactor 1, with whose aid the jacket can be screwed on to the tube plate. For sealing, a sealing element is provided between the flange of the outer jacket and the tube plate, for example a flat seal.

At the opposite end of the tube plate to the outer jacket, a cover of the reactor is generally secured. The cover and the tube plate 33 form the distributor space 3 at one end, and the collection space 5 at the other end. The cover is secured to the tube plate 33 generally with the aid of a screw connection which enables detachment of the cover for the exchange of the catalyst. Between the cover and the tube plate, preference is given to using a sealing element which is suitable for applications in the intended pressure range. The sealing element is preferably a spring-elastic metal seal, a so-called Helicoflex seal, which is inserted into a groove which has been turned in the cover. Alternatively, the sealing can also be effected without a sealing element, in which case obliquely positioned wedges should be incorporated into cover and tube plate, which wedge together to form a sealed system when the screws present between cover and tube plate are tightened.

### LIST OF REFERENCE NUMERALS

1 Reactor
3 Distributor space
5 Collection space
7 Feed
9 Outlet
11 Temperature control circuit
13 Reservoir vessel
15 Feed line
17 Pump
19 Outlet
21 Vapor line
23 Valve
25 Liquid level
31 Tube
33 Tube plate
35 Weld seam
37 Passage orifice
39 Outer space
41 Plating
d Thickness of the tube plate 33

1.-26. (canceled)

27. A reactor for performing high-pressure reactions, the reactor being designed for a pressure range from 100 to 325 bar and comprising at least one tube (31) having each end pass through and bonded to one of a plurality of tube plates (33), the tube plates (33) and the at least one tube (31) being surrounded by an outer jacket, such that an outer space (39) is formed between the tube (31) and the outer jacket, wherein the tube plates (33) have at least one surface composed of a nickel-base alloy and the at least one tube (31) is welded on to the surface composed of the nickel-base alloy, the surface composed of the nickel-base alloy facing in the direction of a respective nearest end of the reactor, and the outer jacket has a thickness which is sufficient to absorb tensile forces which occur between the at least one tube (31) and the outer jacket owing to a temperature difference caused by differences in expansion.

28. The reactor according to claim 27, wherein the nickel-base alloy is applied to the tube plates (33) as a plating (41).

29. The reactor according to claim 28, wherein the plating (41) has a thickness of up to 30 mm.

30. The reactor according to claim 27, wherein the tube plates (33) have a diameter of up to 2,400 mm and a thickness (d) of up to 600 mm.

31. The reactor according to claim 27, wherein the at least one tube (31) has a length in the range from 3,000 to 18,000 mm.

32. The reactor according to claim 27, wherein the at least one tube (31) is manufactured from an austenitic material.

33. The reactor according to claim 27, further comprising thermocouples arranged on the outer jacket and inside the at least one tube (31).

34. The reactor according to claim 27, wherein the outer space (39) is connected to a temperature control medium circuit (11), said temperature control medium circuit (11) comprising a reservoir vessel (13) for a temperature control medium, the reservoir vessel being arranged at a height such that the temperature control medium can flow through the outer space (39) of the reactor owing to the hydraulic pressure of the liquid.

35. The reactor according to claim 34, wherein the temperature control circuit comprises a pump (17) configured as a free-running pump.

36. The reactor according to claim 34, wherein internals are arranged in the outer space (39) to adjust the flow of the temperature control medium.

37. The reactor according to claim 36, wherein the internals are perforated plates.

38. The reactor according to claim 27, wherein the reactor is a tube bundle reactor.

39. The reactor according to claim 38, wherein internals are present in the intake region of the tubes (31) of the tube bundle reactor in order to distribute reactants supplied uniformly between the tubes (31).

40. A process for starting up a reactor according to claim 27, the at least one tube (31) being filled with a catalyst as a reactor bed which is activated by a hydrogenation with hydrogen, and the outer space (39) being filled with water, the process comprising:
a. heating the catalyst to a temperature in the range from 120 to 170° C. at a pressure in the range from 120 to 170 bar in the presence of a nitrogen atmosphere at a rate of from 5 to 15 K/h and simultaneously increasing the temperature of the water in the outer space (39) by supplying steam and increasing the pressure, such that the boiling point of the water in the outer space corresponds to the temperature inside the tube (31),
b. supplying hydrogen until a concentration of hydrogen of from 1 to 3% by volume has been attained and holding the atmosphere for a period of from 5 to 8 h, then increasing the hydrogen concentration to from 4 to 6% by volume and holding the atmosphere for a period of from 5 to 8 h,
c. increasing the hydrogen concentration to from 8 to 12% by volume and holding the hydrogen concentration until the temperature in the reactor bed remains essentially constant, then increasing the hydrogen concentration to from 45 to 55% by volume,
d. increasing the pressure inside the at least one tube (31) to from 150 to 280 bar and increasing the temperature of the hydrogen-containing gas passed through the tubes (31) to from 200 to 230° C. at a rate of from 5 to 15 K/h and increasing the temperature in the outer space (39) by supplying steam and increasing the pressure, such that the boiling point of the water in the outer space (39) corresponds to the temperature in the tube (31),
e. replacing the water-steam mixture in the outer space (39) with dry saturated water vapor,
f. increasing the temperature in the tube interior to from 250 to 300° C. at a rate of from 2 to 8 K/h and holding the temperature for a period of from 20 to 30 h,
g. lowering the temperature in the tube interior at a rate of from 5 to 15 K/h and simultaneously lowering the temperature in the outer space (39) by lowering the pressure.

41. The process according to claim 40, wherein activation of the catalyst is preceded by performance of cleaning of the outer space (39).

42. The process according to claim 41, further comprising:
1. filling the outer space (39) with deionized water, seeding the water with from 0.001 to 0.004 kg of a passivating agent per kg of water, heating to from 110 to 150° C. at a rate of from 5 to 15 K/h, circulating the solution over a period of from 20 to 30 h, cooling at a rate of from 5 to 15 K/h to a temperature in the range from 90 to 110° C., and discharging the solution by supplying an inert gas,
2. filling the outer space (39) with deionized water having a temperature in the range from 80 to 100° C., seeding with from 0.0005 to 0.004 kg of a passivating agent per kg of water, heating to from 110 to 150° C. at a rate of from 5 to 15 K/h, circulating the solution over a period of from 20 to 30 h, cooling at a rate of from 5 to 15 K/h to a temperature in the range from 90 to 110° C. and discharging the solution by supplying an inert gas,
3. repeating step (2) as appropriate until the concentration of iron ions in the solution at the end of the circulation exhibits an asymptotic profile,
4. flushing the outer space with deionized water having a temperature of from 70 to 100° C. for a period of from 0.5 to 2 h,
5. repeating step (4) as appropriate until an electrical conductivity of the water at the end of the flushing operation of not more than 20 μS/cm is measured.

43. A process for performing an exothermic reaction in a reactor according to claim 27, in which at least one reactant as the reaction medium is added to the at least one tube (31) and reacts in the tube (31) at least partly to give a product, and a temperature control medium is added to the outer space (39) and the temperature control medium evaporates by absorbing heat at essentially constant temperature, such that the reaction is performed under essentially isothermal conditions.

44. The process according to claim 43, wherein the temperature control medium and the reaction medium are conducted through the reactor in cocurrent.

45. The process according to claim 43, wherein, in the event of outage in the power supply, temperature control medium from the reservoir vessel (13) is passed through the outer space (39) of the reactor owing to the hydraulic pressure.

46. The process according to claim 45, wherein the pressure in the outer space (39) is lowered.

47. The process according claim 43, wherein the reaction is performed at a temperature in the range from 130 to 300° C.

48. The process according to claim 43, wherein the reactants added are diethylene glycol and ammonia, which are converted to aminodiglycol and morpholine.

49. The process according to claim 43, wherein the reactants used are polyether alcohols and ammonia, which are converted to the corresponding polyetheramines.

50. The process according to claim 43, wherein the reactants used are ethanol, propanols or butanols and ammonia, which are converted to the corresponding ethylamines, propylamines or butylamines.

51. The process according to claim 43, wherein the pressure in the outer space (39) of the reactor is in the range from 4.76 to 86 bar (abs).

52. The process according to claim 43, wherein the temperature control medium used is water, a water-alcohol mixture or a thermal oil.
